(19) United States
(12) Patent Application Publication
(10) Pub. No.: US 2005/0159596 A1
(43) Pub. Date: Jul. 21, 2005

(54) Cyclic compounds and their use as complex ligands

(75) Inventor: Erich Tauer, Gottingen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Basf Aktiengesellschaft, Ludwigshafen (DE)

(21) Appl. No.: 10/503,587

(22) PCT Filed: Feb. 14, 2003
(86) PCT No.: PCT/EP03/01490

(30) Foreign Application Priority Data
Feb. 15, 2002 (DE) 102 06 366.4

(51) Int. Cl. C07D 491/14; C07F 15/00

(52) U.S. Cl. 540/465; 540/468; 540/472

(57) ABSTRACT

A cyclic compound of the general formula (I) 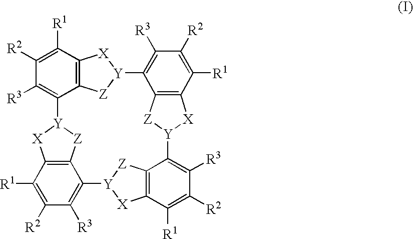 where X-Y-Z is N═C—O, NH—C═N or N═C—NH and
R¹, R² and R³ are each H or C₁₋₆-alkyl
   and of the general formula (III) 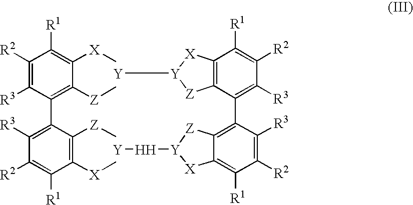 where X-Y-Z is N═C—O and
R¹, R² and R³ are each H or C₁₋₆-alkyl, may be used as complexing ligands.

The present invention relates to cyclic compounds, processes for preparing them, their use as complexing ligands and complexes containing them.

Crown ethers may be used as multidentate complexing agents. Crown ethers are a class of planar macrocyclic polyethers. The oxygen atoms are frequently bridged by ethylene bridges, and in many cases one or more benzene or cyclohexane rings are fused on. The oxygen atoms of the crown ethers may also be partly or fully replaced by other heteroatoms such as nitrogen, phosphorus or sulfur. This results in, for example, aza-, phospha- or thia-crown ethers. Polar groups may also be present which may occupy the donor positions.

The crown ethers known hitherto do not have an entirely suitable property profile for all complexing tasks. There is accordingly still a demand for cyclic complexing ligands which have novel property profiles.

It is an object of the present invention to provide cyclic compounds which may preferably be used as complexing ligands.

We have found that this object is achieved by a cyclic compound of the general formula (I) 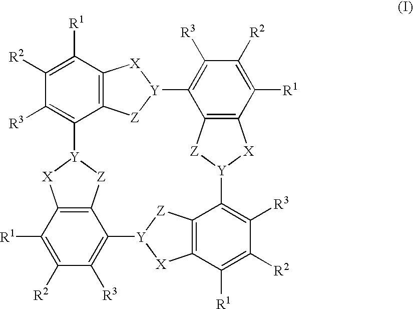
where
X-Y-Z is N═C—O, NH—C═N or N═C—NH and
R¹, R² and R³ are each H or C₁₋₆-alkyl.

We have found that this object is also achieved by a cyclic compound of the general formula (III) 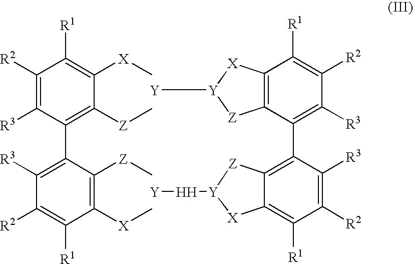
where
X-Y-Z is N═C—O
R¹, R² and R³ are each H or C₁₋₆-alkyl.

When X-Y-Z has the meaning NH—C═N or N═C—NH, it should be noted that the structures are tautomeric.

The R¹, R² and R³ radicals may each be selected in such a manner that they do not hinder the cyclization reaction for preparing the compounds of the general formula (I). The radicals are preferably each independently hydrogen atoms or C₁₋₃-alkyl radicals, more preferably hydrogen atoms or methyl radicals, in particular hydrogen atoms.

X-Y-Z and R¹, R² and R³ all have the same definition for all positions.

The cyclic compounds of the general formula (I) can be prepared by cyclizing compounds of the general formula (II) 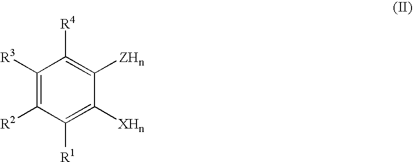
where
R⁴ is —COOH or a derivative thereof,
each n is 1 or 2, to maintain the stoichiometry, and
R¹, R² and R³ are each H or C₁₋₆-alkyl.

The R⁴ radical is a carboxylic acid radical or a derivative thereof. The derivative is preferably an acid chloride, an ester, an amide or another appropriate carboxylic acid derivative. The esters are preferably esters of lower alkanols such as C₁₋₄-alkanols. The amides are preferably derived from ammonia or primary alkylamines.

The cyclization is carried out preferably by heating in polyphosphoric acid. In general, operation is effected under a protective gas atmosphere (nitrogen).

Preference is given to preparing the compounds of the general formula (III) by photochemical reaction of smaller precursor compounds. This is illustrated hereinbelow using a preferred compound.

The cyclic compounds of the general formulae (I) and (III) may be used for a variety of applications. Preference is given to using them as complexing ligands. The invention also relates to corresponding complexes comprising a complexed metal ion and at least one cyclic compound of the general formulae (I) and (III) as a complexing ligand.

Preferred metal ions to be complexed are derived from alkali metals.

In addition to the cyclic compounds according to the invention, there may be further ligands which are customary in the field of complex chemistry.

The compound (5) has the form of a stiff and flat ring and is similar to a 16-crown-4-ether. At a first glance, compound (8) is similar to a porphyrin system. However, the imidazoles are tautomeric so that the hydrogen atom on the nitrogen may move from position 1 to position 3 and back. This means that a metal atom in the center of compound (8) may be bonded by 4, 3, 2 or one covalent bond or without covalent bonds. In contrast, there are always only two delocalized covalent bonds in porphyrin, phthalocyanine and similar systems.

The preparation of the cyclic compounds according to the invention is illustrated with reference to the reaction schemes hereinbelow.

The most general process for preparing 2-R-benzoxazoles comprises the condensation of a 2-aminophenol with a suitable carboxylic acid, acid chloride, ester, amide or other carboxylic acid derivative. For example, cyclic quaternization of 3-amino-2-hydroxy-benzamide (4) may be carried out in order to obtain the desired product (5). The amide (4) can be prepared from the precursor 2-hydroxy-3-nitrobenzamide (3). By heating (4) in polyphosphoric acid (ppa) under nitrogen, the desired compound (5) may be obtained in acceptable yields. In the case of this particular compound (5), the yield was 32% and the compound forms colorless crystals having an unexpectedly high melting point of 520° C. The substance has very limited solubility in all organic solvents. The compound has the expected UV spectrum which has a maximum for the long wavelengths at 364 nm in CHCl₃.

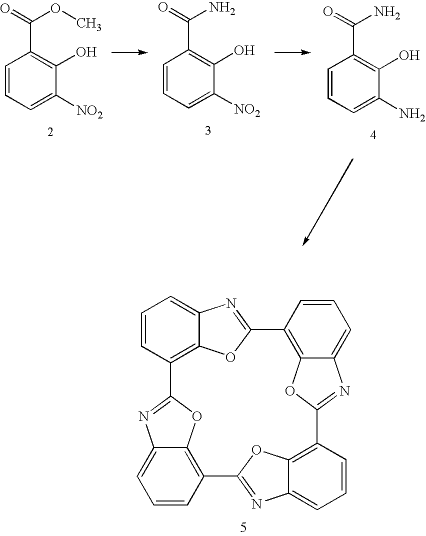

Cyclo-2,4′:2′,7″:2″4′″:2′″,7-quaterbenzimidazole (8) may be prepared in a similar manner to the preparation of (5). To this end, for example, 2-amino-3-nitrobenzoic acid (6) may be catalytically reduced in dilute ammonia using H₂/Pd to obtain an ammonium salt of 2,3-diaminobenzoic acid (7). This acid (7) may then be heated in polyphosphoric acid to obtain the cyclic compound (8). For the particular compound (8), a nonoptimized yield of 14.5% was obtained. The compound (8) forms a yellow microcrystalline powder which has an even lower solubility in organic solvents than compound (5). Compound (8) may be sublimed at 630° C. and 6×10⁻³ hPa. At atmospheric pressure under nitrogen, the color of the compound changes from yellow to brown at a temperature from 700° C. to 750° C. Partial sublimation but no melting is observed.

The ¹H NMR spectrum allows the unambiguous assignment of the aromatic protons of the structure (8). The long wavelength maximum in the UV-VIS spectrum is at 393 nm (in DMSO) for compound (8).

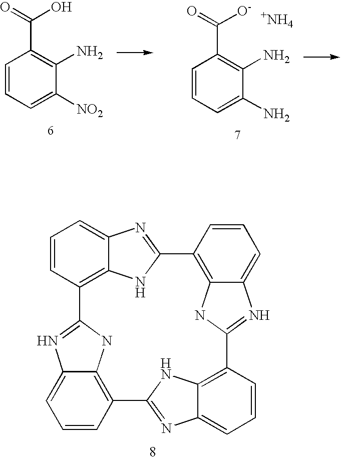

Benzoxazole may be symmetrically photodehydrodimerized to form 2,2′-dibenzoxacolyl. This conversion is effected under a radiation in aerated polar solvents.

7,7′:2′,2″:7″,7′″-Quaterbenzoxazole (13) may be prepared by irradiating a solution of (12) in methanol using light of a wavelength 245 nm. Compound (13) precipitates out of solution in the form of colorless crystals having analytical purity. The solubility of compound (13) in methanol, ethanol and acetonitrile is <10⁻⁶ moll. The solubility is improved in chloroform and dimethylformide.

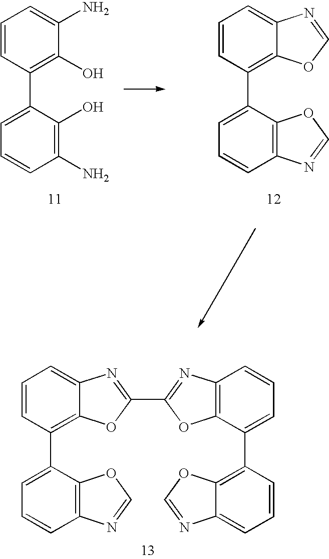

The above-described synthesis of compound (5) allows linking of the individual benzoxazole units between positions 2 and 7. Appropriate synthetic processes using different starting materials may also be used to prepare isomeric molecules of the type of compound (5) where one or more units are linked between positions 2 and 4. This leads to the nitrogen atoms facing toward the center of the molecule. Compounds (5) and (13) may also be replaced by appropriate compounds where the benzoxazole units are replaced by other benzo-x-azoles (8).

The invention is illustrated by the examples hereinbelow.

### EXAMPLES

The following analytical instruments were used:

UV: Perkin-Elmer 320

¹H NMR: Bruker WP 80 CW, Varian Unity Plus 500 [internal standard TMS and CDCl₃].

MS: Varian MAT CH 17

Melting points: uncorrected.

2-Hydroxy-3-nitrobenzamide (3)

19.7 g (0.1 mol) of methyl 2-hydroxy-3-nitrobenzoate (2) were suspended in 250 ml of ammonia (25% in water) and stirred at room temperature for 3 days. After 30 hours, all of compound (2) was dissolved. The solvent was evaporated and two 20.9 g of a red residue, the ammonium salt of (3), was retained. 200 ml of water and 9 g (0.15 mol) of acetic acid were added and the mixture was stirred for 5 hours. The precipitated product was filtered off using a suction filter and washed with water and dried. The yield was 17.2 g (94.5%) of compound (3) whose melting point was 149 to 150° C. The product was used without further purification steps.

The compound used (2) may be prepared from the free acid as described in J. Het. Chem. 1971, 8, pages 989 to 991. This reference describes both the preparation of esters and amides. The conversion of a nitro compound to the amino compound is also described. This reference therefore also describes further possible analogs of compounds (2), (3) and (4).

3-Amino-2-hydroxybenzamide (4)

17.2 g (94.5 mmol) of compound (3) were dissolved in 350 ml of ethyl acetate. 1.7 g of Pd (10% on carbon) were installed and the nitro group was reduced using hydrogen at atmospheric pressure and room temperature. The catalyst was filtered off and washed with ethyl acetate, and the filtrate was concentrated by evaporation. The yield was 14.5 g (98%) of compound (4) which had a melting point of 127 to 129° C. and was clean enough for the next reaction stage. An analytical sample was chromatographed (SiO₂-ethyl acetate) and recrystallized from toluene. Colorless crystals having a melting point of 134 to 135° C. were obtained.

UV (2-propanol): λₘₐₓ(1 g ε)=330 nm (3.55), 266 (3.44), 260 (3.53), 253 (3.59).

¹H NMR ([d₈]THF): δ=4.2 (br.s, 2H, amine-H), 6.4-6.95 (m, 3H, arom. H), 7.15 (br.s, 2H, amide H), 13.1 (br.s, 1H, OH).

Molecular weight 152 (MS).

C₇H₈N₂O₂ (152.15): calculated C, 55.26; H, 5.30; N, 18.41; found C, 55.56; H, 5.24; N, 18.11.

Cyclo-2,7′:2′,7″:2′″,7′″:2′″,7-quaterbenzoxazole (5)

4 g (26 mmol) of compound (4) in 120 g of polyphosphoric acid (ppa) were heated under nitrogen to 220° C. and stirred for 24 hours. After cooling, the reaction mixture was poured onto about 1 kg of ice and neutralized using ammonia (25% in water) to a pH of from 6 to 7. After being left to stand overnight, the precipitate was filtered off with suction, washed thoroughly with water and dried. The product (3.22 g) was extracted in a jacketed Soxhlet extractor using 250 ml of pyridine for 9 days. Compound (5) separated from the solution as a gray-white powder and the yield was 0.98 g (32%). Recrystallization from quinoline and subsequent sublimation (490° C., 5×10⁻³ hPa) gave the pure compound (5), colorless microcrystals having a melting point of 517 to 520° C. (without decomposition). The melting point was determined in sealed capillaries having an internal pressure of 200 hPa of nitrogen.

UV (CHCl₃): λₘₐₓ (1 g ε)=346 nm (4.51), 328 (4.75), 315 (4.73), 302 (4.76), 276 (4.58).

¹H NMR (CDCl₃, 50° C.): δ=7.57 (tr, J=8, 4H), 8.00 (d, J=8,4H), 8.54 (d, J=8,4H) Molecular weight 468 (MS).

C₂₈H₁₂N₄O₄ (468.42): calculated C, 71.79; H, 2.58; N, 11.96; found C, 71.59; H, 2.68; N, 11.90.

Cyclo-2,4′:2′,7″:2″4′″:2′″,7-quaterbenzimidazole (8)

9.1 g (50 mmol) of 2-amino-3-nitrobenzoic acid (6) were dissolved in 300 ml of ammonia (5% in water). 1 g of Pd (10% on carbon support) was added and the substance was reduced using hydrogen at room temperature and atmospheric pressure. The catalyst was filtered off with suction and the solution evaporated to dryness. The ammonium salt of 2,3-diaminobenzoic acid (7, 8.28 g, 98% yield) obtained was used without further purification.

4 g (24 mmol) of compound (7) in 125 g of polyphosphoric acid (ppa) were heated to 220° C. and stirred under nitrogen for 0.20 hours. After cooling, the product was poured onto about 1 kg of ice and neutralized using ammonia (25% in water) to a pH of from 7 to 8. The very fine precipitate was filtered off with suction, washed thoroughly with water and dried. The yield was 2.8 g. The product was extracted in a jacketed Soxhlet extractor using 250 ml of pyridine for 12 days. Compound (8) separated from the solvent as green-gray microcrystals, and the yield was 396 mg (14.5%). The product was sublimed by sublimation at 630° C. and 6×10⁻³ hPa to obtain a yellow-green product. The substance gradually darkened at a temperature of from 700 to 750° C. without melting (when heating under nitrogen and at atmospheric pressure), and the process was accompanied by partial sublimation.

UV(DMSO):λₘₐₓ(1 g ε)=393 nm (3.99), 372s (4.24), 356s (4.78), 349 (4.82), 340 (4.87), 284(4.66).

¹H NMR ([d₆]-DMSO, 120° C.): δ=7.51 (tr, 2H, J=8), 7.53 (tr, 2H, J=8), 7.79 (d, 2H, J=8), 7.96 (d, 2H, J=8), 8.23 (d, 2H, J=8), 8.40 (d, 2H, J=8). No signals of NH protons could be detected. Molecular weight 464 (MS).

C₂₈H₁₆N₈ (464,48): calculated C, 72.40; H, 3.47; N, 24.12; found C, 72.51; H, 3.56; N, 24.05.

2-Amino-3-nitrobenzoic acid may be prepared as described in J. Med. Chem. 1990, 33, pages 814 to 819. This document also describes the further reaction to substituted benzimidazoles, see the reaction schemes on page 815. Substituted 2,3-diaminobenzoic acids are described on page 816 in scheme II. Possible substituents are also stated in table II so that this document also describes the preparation of similar, substituted compounds.

7,7′-Bibenzoxazolyl (12)

6.85 g (32 mmol) of 3,3′-diaminobiphenyl-2,2′-diol (11) and 46 g (1 mol) of formic acid were heated to reflux for 3 hours. The acid was distilled off and the residue heated to 180° C. for two hours under nitrogen. After cooling, the product was washed with water and dried. Chromatographic purification (SiO₂, ethyl acetate) and recrystallization from methanol gave 2.77 g (37%) of the pure compound (12) in the form of slightly ochre-colored crystals which had a melting point of 226 to 228° C.

UV (2-propanol): λₘₐₓ (1 g ε)=265 nm (4.27).

¹H NMR (CDCl₃): δ=7.4-8.0 (m, 6H, arom. H), 8.2 (s, 2H, CH).

Molecular weight 236 (MS).

C₁₄H₈N₂O₂ (236.22): calculated C, 71.18; H, 3.41; N, 11.86; found C, 71.12; H, 3.56; N, 11.78.

3,3′-Diaminobiphenyl-2,2′-diol may be prepared as described in Ber. 1902, 35, pages 302 to 313. Differently substituted compounds of this type and the preparation thereof are also described in this reference.

7,7′:2′,2″:7′″,7′″-Quaterbenzoxazole (13)

1 g (4.25 mmol) of compound (12) in 1.3 l of aerated methanol were irradiated with light from a 30 W low pressure mercury lamp (Original Hanau, NN 30/89, wavelength 254 nm, used as a submerged lamp). The irradiation was carried out for 2 days. During the irradiation, compound (13) precipitated, and was filtered off, washed and dried. The yield was 180 mg (18%) of colorless crystals which had a melting point of 358 to 362° C.

UV (CHCl₃): λₘₐₓ (1 g ε)=347 nm (4.29), 330 (4.49), 320 (4.47)

¹H NMR (C₂D₂Cl₄, 120° C.): δ=7.6-8.1 (m, 12H, arom. H), 8.2 (s, 2H, CH). Molecular weight 470 (MS).

C₂₈H₁₄N₄O₄ (470.44); calculated C, 71.49; H, 3.00; N, 11.91); found C, 71.39; H, 2.93; N, 11.93.

1-9. (canceled)

10. A cyclic compound of formula (I) 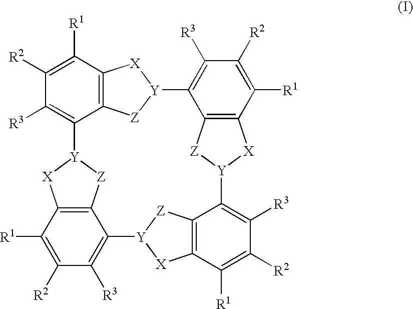
where
X-Y-Z is N═C—O,
R¹, R²
and R³ are each H, C₁₋₆-alkyl or
X-Y-Z is NH—C═N or N═C—NH and
R¹, R² and R³ are each C₁₋₆-alkyl.

11. A cyclic compound as claimed in claim 10, wherein R¹, R² and R³ are each H or C₁₋₃-alkyl, when X-Y-Z is N═C—O, or are each C₁₋₃-alkyl, when X-Y-Z is NH—C═N or N═C—NH.

12. A cyclic compound as claimed in claim 11, wherein R¹, R² and R³ are each hydrogen, when X-Y-Z is N═C—O.

13. A process for preparing cyclic compounds of formula (I) as claimed in claim 10, which comprises:
cyclizing compounds of formula (II) 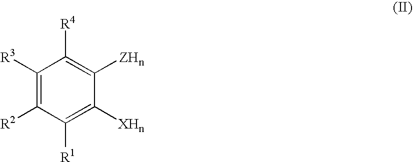
where
R⁴ is —COOH or a derivative thereof,
X and Z are independently N or O, wherein each n is 1 or 2 to maintain the stoichiometry, and
R¹, R² and R³ are each H or C₁₋₆-alkyl, and.

14. A process as claimed in claim 13, wherein the cyclization is carried out by heating in polyphosphoric acid.

15. A process for preparing cyclic compounds of formula (I) 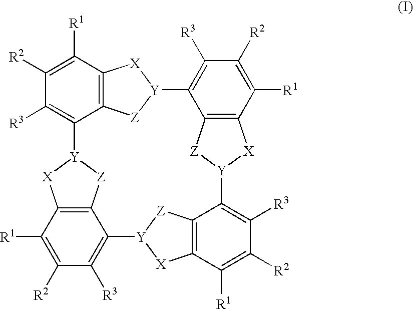
where
X-Y-Z is NH—C═N N═C—NH
R¹, R², R³ H,
which comprises:
cyclizing compounds of formula (II) 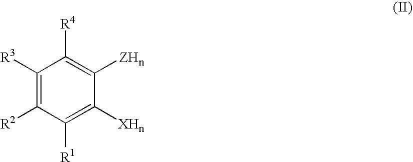
where
R⁴ is COOH or a derivative thereof,
X and Z are independently N or O, wherein each n is 1 or 2 to maintain the stoichiometry, and
R¹, R², R³ H,
wherein the cyclizing occurs by heating in polyphosphoric acid.

16. A complex comprising a complexed metal ion and at least one cyclic compound as defined in claim 10 as a complexing ligand.

17. A cyclic compound of formula (III) 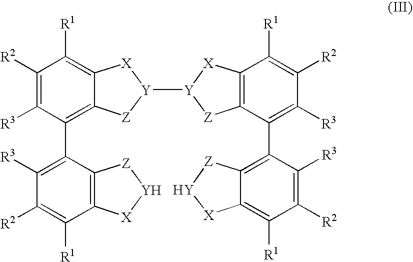
where
X-Y-Z is N═C—O and
R¹, R² and R³ are each H or C₁₋₆-alkyl.
